(19) United States
(12) Patent Application Publication
(10) Pub. No.: US 2025/0082246 A1
(43) Pub. Date: Mar. 13, 2025

(54) DEVICE FOR PROCESSING INTRACARDIAC SIGNALS

(71) Applicant: SUBSTRATE HD, Marseille (FR)

(72) Inventor: Thomas BOUDOU, Marseille (FR)

(73) Assignee: SUBSTRATE HD, Marseille (FR)

(21) Appl. No.: 18/723,705

(22) PCT Filed: Dec. 23, 2022
(86) PCT No.: PCT/FR2022/052497

§ 371 Date: Jun. 24, 2024

(30) Foreign Application Priority Data
Dec. 24, 2021 (FR) FR2114503

(51) Int. Cl. A61B 5/349; G16H 40/60

(52) U.S. Cl. CPC A61B 5/349; G16H 40/60

(57) ABSTRACT

The invention relates to a device for processing intracardiac signals, which comprises a memory unit (4) arranged to receive electrocardiogram data and synchronised electrogram data, a detector (6) arranged to analyse the electrocardiogram data and to detect QRS wave instants therein, an analyser (8) arranged to perform a wavelet transform of the electrogram data, an extractor (10) arranged to collect coefficients from the wavelet transform, each associated with a QRS wave instant detected by the detector (6), and to store them in a buffer (14), and a composer (12) arranged to extract a QRS fingerprint signal from the buffer (14) and subtract it from the wavelet transform at the QRS wave instants, and to output denoised electrogram data by inverse wavelet transform of the resulting signal.

The invention relates to the field of processing and denoising intracardiac signals.

The measurement of the atrial electrograms proximate to the ventricles (for example in the vein of the coronary sinus—the reference catheter used during the ablation procedures) may be disturbed by the ventricular activity.

The latter generates waveforms on the intracardiac electrograms also known as “far field” (FF). Such a distortion by the ventricles makes the analysis of the signal of the atrial electrograms more difficult and this information not related to the atria is wrongly included (for example, in the estimate of the length of the cycle).

In particular, the invention aims to cancel the far-field ventricular contribution in the electrogram tracks derived from intracardiac probes, considered as noise, while preserving the near-field activity, even when there is superposition of these.

In clinical practice, i.e. in operating rooms, the influence of the far field is treated by abrupt erasing of the signal where the latter is detected. This is not very effective, and even degrades the signal.

### Scientific Literature Offers Several Axes:

TMS (standing for “Template matching and substraction”), which consists in calculating an average of a QRS complex over the duration of a record, and in subtracting this average from each QRS complex encountered (cf., for example Rieta, J. J., et al. “Atrial activity extraction based on blind source separation as an alternative to QRST cancellation for atrial fibrillation analysis.” Computers in Cardiology 2000. Vol. 27 (Cat. 00CH37163), IEEE, 2000),
independent component analysis (ICA), which consists in finding a set of components that minimises the mutual information existing in the ECG signal at different intervals. The independent components may be grouped in the subspaces of ventricular activity and of atrial activity, which allows reconstructing the atrial activity at each observation point from the atrial activity subspace (cf., for example, the article by F. Castells, et al. “Multidimensional ICA for the Separation of Atrial and Ventricular Activities from Single Lead ECGs in Paroxysmal Atrial Fibrillation Episodes”, Lecture Notes in Computer Science, vol. 3195, p. 1229-1236, 2004),
adaptive ventricular cancellation (AVC), which consists in using a finite impulse response filter on the reference channel in order to estimate the interference, then removing these from the reference channel (cf., for example, the article by Widrow B., et al. “Adaptive noise cancelling: principles and applications”, Proc. IEEE 63, 1692-716, 1975),
coupling the ICA with a wavelet decomposition, which consists in applying the ICA on the wavelet decompositions of the electrograms and of the electrocardiogram (cf., for example, the article by Simanto Saha, et al. “A Ventricular Far-field Artefact Filtering Technique for Atrial Electrograms”, 2019 Computing in Cardiology Conference, 2019, and
principal component analysis (PCA), which consists in transforming variables related to one another (so-called “correlated” in statistics) into new variables decorrelated from one another (cf., for example, the article by Christopher Schilling, “Analysis of Atrial Electrograms”, Vol. 17 Karlsruhe Transactions on Biomedical Engineering, 2012.

All these methods have considerable disadvantages. Thus, TMS deforms the atrial electrograms, which makes unusable any method that uses it as a base, the ICA loses a large part of its effectiveness as soon as the signals become irregular and disorganised, the AVC is less effective than the ICA and, likewise, the effectiveness of the cancellation considerably depends on the reference signal, and the PCA improves the TMS, but with similar defects.

Hence, no method allows effectively denoising the electrogram signals to enable exploitation thereof in an atria arrhythmia context.

The invention improves the situation. To this end, it provides an intracardiac signal processing device, comprising a memory arranged to receive electrocardiogram data and synchronised electrogram data, a detector arranged to analyse the electrocardiogram data and to detect therein QRS wave time points, an analyser arranged to carry out a wavelet transform of the electrogram data, an extractor arranged to derive in the wavelet transform coefficients each associated with a QRS wave time point detected by the detector, and to store them in a buffer, and a composer arranged to extract from the buffer a QRS fingerprint signal and subtract it in the wavelet transform at the QRS wave time points, and to produce as output denoised electrogram data by inverse wavelet transform of the resulting signal.

This device is particularly advantageous because it allows not only cancelling the activity of the far field, but also reconstructing the near field where it is superposed with the far field, thereby considerably increasing the signal-to-noise ratio.

According to various embodiments, the invention may have one or more of the following features:
the memory is arranged to receive electrogram data which correspond to distinct tracks, the detector, the analyser, the extractor and the composer being arranged to independently process the electrogram data associated with distinct tracks,
the extractor is arranged to derive coefficients such that, for a selected coefficient corresponding to a given time point in a given wavelet transform level, the wavelet signal which corresponds to the given wavelet level and which is centred on the given time point has an overlap with a window extracted from the electrogram data from which the selected coefficient is derived, which window is centred on the QRS wave time point with which each respective coefficient is associated,
the extractor is arranged to weight the coefficients stored in the buffer according to the temporal overlap between the wavelet signal which corresponds to the wavelet level of each respective coefficient and which is centred on the time point corresponding to each respective coefficient and the window centred on the QRS wave time point with which each respective coefficient is associated,
the composer is arranged to define a QRS fingerprint signal for each wavelet level of the wavelet transform, each based on a function of the coefficients derived by the extractor for a respective wavelet level,
the composer is arranged to apply a function selected from the group including the geometric median, the PCA or the ICA,
the composer is arranged to use a selected number of most recent coefficients in the buffer for each wavelet level, and
the extractor is arranged to carry out a SWT-type wavelet transform.

The invention also relates to a method for processing intracardiac signals comprising:
a) receiving electrocardiogram data and synchronised electrogram data,
b) analysing the electrocardiogram data to detect therein QRS wave time points,
c) carrying out a wavelet transform of the electrogram data,
d) deriving in the wavelet transform coefficients, each associated with a QRS wave time point detected in the operation b), and storing them in a buffer,
e) extracting from the buffer a QRS fingerprint signal and subtracting it in the wavelet transform of the operation c) at the QRS wave time points, and
f) carrying out an inverse wavelet transform of the signal of the operation e), and returning as output the corresponding denoised electrogram data.

According to various embodiments, this method may have one or more of the following features:
the operation d) comprises deriving coefficients from the wavelet transform of the operation c) such that, for a selected coefficient corresponding to a given time point of a given level of the wavelet transform, the wavelet signal which corresponds to the given level of the wavelet transform and which is centred on the given time point has an overlap with a window extracted from the electrogram data from which the selected coefficient centred on the QRS wave time point with which the selected coefficient is associated, is derived,
the operation d) comprises weighting each derived coefficient before storing it in the buffer, according to the temporal overlap between the wavelet signal which corresponds to the level of the wavelet transform of each respective derived coefficient and which is centred on the time point corresponding to each respective derived coefficient and the window centred on the QRS wave time point associated with each respective derived coefficient,
the operation e) comprises defining a respective QRS fingerprint signal for each wavelet level of the wavelet transform of the operation b), each based on a function of the coefficients of the operation d) corresponding to a respective wavelet level,
the function is selected from the group including the geometric median, the PCA or the ICA,
the function uses a selected number of most recent coefficients in the buffer for each QRS fingerprint signal, and
the operation c) carries out a SWT-type wavelet transform.

The invention also relates to a computer program comprising instructions for executing the method according to the invention, a data storage medium on which such a computer program is recorded and a computer system comprising a processor coupled to a memory, the memory having recorded such a computer program.

Other features and advantages of the invention will appear better upon reading the following description, derived from examples given for illustrative and non-limiting purposes, with reference to the drawings wherein:

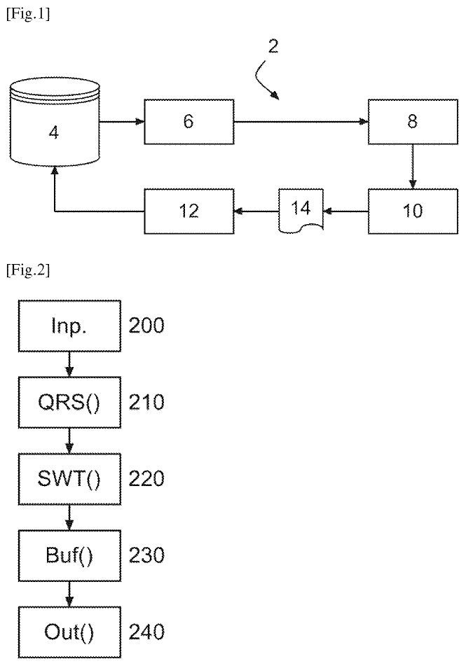

FIG. 1 shows a generic diagram of a device according to the invention,

FIG. 2 shows an embodiment of a function implemented by the device of FIG. 1,

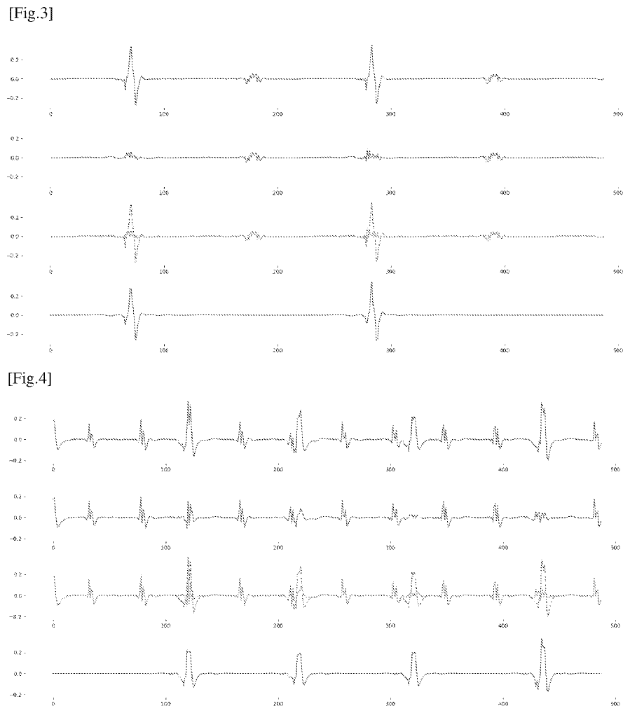

FIG. 3 shows an example of an electrogram signal before denoising, after denoising, the superposition as well as the difference between these signals, and

FIG. 4 shows another example of an electrogram signal before denoising, after denoising, the superposition as well as the difference between these signals.

The drawings and the description hereinafter essential contain elements of a certain nature. Hence, they could not only be used to better understand the present invention, but also contribute to the definition thereof, where appropriate.

FIG. 1 shows a generic diagram of an intracardiac signal processing device 2 according to the invention. The device 2 comprises a memory 4, a detector 6, an analyser 8, an extractor 10, a composer 12 and a buffer 14.

The memory 4 may consist of any type of data storage capable of receiving digital data: hard disk, solid-state drive, flash memory in any form, random-access memory, magnetic disk, storage distributed locally or in the cloud, etc. The data calculated by the device may be stored on any type of memory similar to the memory 4, or on the latter. These data may be erased after the device has performed its tasks or preserved.

The memory 4 receives various types of data:
electrogram data, which represent the signal measured on one or more track(s) of a cardiac catheter. When these data originate from distinct tracks, they are arranged so that the data associated with each distinct track can be grouped together. For example, it is conventional that 5 distinct tracks are received,
electrocardiogram data,
QRS wave time point data, the determination of which will be explained hereinbelow,
QRS fingerprint data, the determination of which will be explained hereinbelow, and
denoised electrogram data.

The buffer 14 may be made as a subset of the memory 4, or be separated from the latter. It may be implemented with the same means as those described hereinabove with reference to the memory 4.

The device 2 exploits the diversity and the complementarity of the signals available in an electrophysiology procedure.

As will be seen hereinbelow, the detector 6 first determines the position of the far-field QRS wave time points on the electrocardiogram. Indeed, these are aligned with the noise of the ventricular activity in the intracardiac branches. Afterwards, this noise is cancelled from the other electrograms without suppressing the near-field activity, including when there is superposition of the far field and of the near field, thanks to a time-frequency analysis.

The analyser 8 and the extractor 10 implement a time-frequency approach to separate the far-field components, of ventricular origin, from the bipolar electrograms recorded locally, i.e. the near field.

Simultaneous recordings of surface electrocardiograms and of endocavitary signals have been used to segment the endocavitary signal into several time-frequency components.

The extractor 10 collects and stores each far-field ventricular activity, specific to each track. These activities are detected thanks to their synchronicity with the QRS wave time points which may be detected in the electrocardiogram data by a thresholding method in a wavelet domain. They are stored in a buffer 14. Alternatively, the Pan-Tompkins algorithm can be implemented to detect the QRS wave time points.

At the beginning, the buffer 14 is empty. Afterwards, it is rapidly filled so that respective fingerprints of each track can be calculated allowing cancelling the far-field activities.

Afterwards, the composer 12 subtracts these fingerprints in the wavelet domain, then reconstitutes the denoised signal by performing an inverse wavelet transform.

The underlying idea is that the atrial and ventricular activities can be considered as statistically independent and originating from two distinct sources.

In this case, the intracardiac branches contain the mixture of atrial and ventricular contents that the wavelet domain allows treating effectively.

The detector 6, the analyser 8, the extractor 10, and the composer 12 directly or indirectly access the memory 4. They may be made in the form of a suitable computer code executed on one or more processor(s). By processors, it should be understood any processor suited to the calculations described hereinbelow. Such a processor may be made in any known manner, in the form of a microprocessor for a personal computer, laptop, tablet or smartphone computer, a dedicated chip of the FPGA or SoC type, a computing resource on a grid or in the cloud, a cluster of graphic processors (GPUs), a microcontroller, or any other form capable of providing the computing power necessary for the implementation described hereinbelow. One or more of these elements may also be made in the form of specialised electronic circuits such as an ASIC. A combination of processors and electronic circuits may also be considered.

FIG. 2 shows an example of implementation of a denoising function implemented by the device 2.

In a first operation 200, the electrogram data and the electrocardiogram data synchronised with one another are received. Typically, these data are received in slices of 2s each.

Afterwards, in an operation 210, the detector 6 executes a function QRS( ) which receives the electrocardiogram data as arguments, and returns QRS wave time points detected therein as described hereinabove.

In parallel, or sequentially, the analyser 8 executes an operation 220 in which a function SWT( ) receives the electrogram data as an argument and returns a plurality of wavelet decompositions, each corresponding to a distinct track from which the electrogram data are derived. In the example described herein, the function SWT( ) implements a stationary wavelet transform type algorithm (SWT), which has the advantage of filling the absence of invariance by translation of the discrete wavelet transform (DWT), and avoid dividing the size of the signal by two at each new decomposition level. Alternatively, a discrete wavelet transform could be used, or a continuous wavelet transform (CWT).

Once the wavelet transforms and the QRS wave time points obtained, the extractor 10 executes a function Buf( ) in an operation 230. The function Buf( ) receives the wavelet transforms and the QRS wave time points and stores in the buffer 14 extracts of these wavelet transforms.

Each wavelet level of each track is stored separately in the buffer 14, so that it is possible, for each extract in the buffer 14, to determine to which track and to which wavelet level it corresponds.

This is important, as the Applicant has discovered that it is interesting to store the wavelet coefficients which correspond to the QRS wave time points of each track so as to be able to remove from the signal the component corresponding to the far field.

More specifically, a QRS window is defined around the QRS wave time point that defines the period during which it is considered that the electrogram signal comprises far-field signal. In the example described herein, this window is centred on each QRS wave time point and lasts 120 ms, which corresponds to a common duration of the QRS waves in medical knowledge. Alternatively, the window could be defined differently, and for example be defined by the function QRS( ).

Each wavelet level is associated with a wavelet function which has a duration related to the used wavelet transform as well as to the wavelet level. Hence, each wavelet coefficient of the wavelet transform is associated with a time window which is centred on the time point with which this coefficient is associated and which has the width of the wavelet function of this wavelet level.

Thus, for a given wavelet level and track, the function Buf( ) selects, at each QRS wave time point, the wavelet transform coefficients whose time window has an overlap with the QRS window.

Optionally, the Applicant has discovered that it was advantageous to weight the wavelet coefficients stored in the buffer 14 according to the amount of overlap between the time window of a given coefficient and the QRS window.

Indeed, for the most extreme time points of the QRS window, the time window has a lower overlap, since it is centred on the considered time point. Yet, since the signal corresponding to this coefficient is intended to be suppressed, there is a risk of suppressing the signal that does not correspond to the far field. Similarly, the high-level wavelets may have a wider time window than the QRS window, which obviously poses similar problems. The weighting corresponding to the relative amount of overlap allows limiting these edge effects.

Still alternatively, a trade-off may consist in limiting the wavelet level so that the widest time window has a size in the same range as that of the QRS window.

Once the buffer 14 is filled enough, for example when the buffer 14 contains at least 5 extracts of the operation 230 for each track, the composer 12 can execute a function Out( ) in an operation 240. In the function Out( ), the composer 12 determines a median for each track and wavelet level of the extracts that relates thereto, and subtracts this median from the wavelet transform of the electrogram data of the track and the corresponding wavelet level. By median, it should be understood any method allowing determining a value representative of the set of extracts, regardless of whether it is obtained by means of a geometric median, a PCA or an ICA. Finally, the function Out( ) executes an inverse wavelet transform of the signal in which the median has been subtracted, and returns the corresponding denoised signal.

FIG. 3 and FIG. 4 allow showing the gain conferred by the device 2, in particular in the areas in which the far field and the near field are superposed. In these figures, the Applicant has represented at the top the signal of the electrogram data (i.e. non-denoised), the denoised signal, the superposition and the difference between these signals which highlight the effect of the subtraction of the QRS fingerprint signals in the inverse wavelet transform.

These figures show the efficiency of the device 2, which completely recreates the near-field signal where there was superposition with the far field, and without degrading the signal outside these areas.

Furthermore, the Applicant has conducted a study in order to quantify the gains obtained thanks to the device 2. For this purpose, the Applicant has compared three methods for analysing their relevance, based on electrograms whose cycle duration is known. For this purpose, the Applicant has taken the corresponding electrogram signals and has created three copies thereof: an unchanged first copy, a second copy in which the signal at the ventricular QRS wave time points is suppressed, i.e. replaced by an isoelectric line preserving the continuity of the signal, and a third copy denoised with the device 2. Afterwards, the resulting signals have been used to determine the duration of the cycle, then compared with the known actual duration of the cycle.

This study has been carried out on a cohort of 52 patients who could be in sinus rhythm, atrial tachycardia or atrial fibrillation. The score that has been calculated for each copy is the root mean square of the error, according to the formula

$$S ( c ) = 100 \% * \sqrt{\frac{1}{n} * {\sum }_{i = 1}^{n} \frac{{\vert {y}_{c} ( i ) - y ( i ) \vert}^{2}}{{y ( i )}^{2}}}$$

Where c is the index of the considered copy, y_{c}(i) is the cycle duration determined based on the i-th electrogram signal of the copy with the index c, and y(i) is the actual cycle duration.

The scores are as follows: 13.36% for the first copy, 8.45% for the second copy, and 4.06% for the third copy.

This demonstrates the signal-to-noise ratio gain obtained thanks to the device 2.

1. An intracardiac signal processing device, comprising a memory arranged to receive electrocardiogram data and synchronised electrogram data, a detector arranged to analyse the electrocardiogram data and to detect therein QRS wave time points, an analyser arranged to carry out a wavelet transform of the electrogram data, an extractor arranged to derive in the wavelet transform coefficients each associated with a QRS wave time point detected by the detector, and to store them in a buffer, and a composer arranged to extract from the buffer a QRS fingerprint signal and subtract it in the wavelet transform at the QRS wave time points, and to produce as output denoised electrogram data by inverse wavelet transform of the resulting signal.

2. The device according to claim 1, wherein the memory is arranged to receive electrogram data which correspond to distinct tracks, the detector, the analyser, the extractor and the composer being arranged to independently process the electrogram data associated with distinct tracks.

3. The device according to claim 1, wherein the extractor is arranged to derive coefficients such that, for a selected coefficient corresponding to a given time point in a given wavelet transform level, the wavelet signal which corresponds to the given wavelet level and which is centred on the given time point has an overlap with a window extracted from the electrogram data from which the selected coefficient is derived, which window is centred on the QRS wave time point with which each respective coefficient is associated.

4. The device according to claim 3, wherein the extractor is arranged to weight the coefficients stored in the buffer according to the temporal overlap between the wavelet signal which corresponds to the wavelet level of each respective coefficient and which is centred on the time point corresponding to each respective coefficient and the window centred on the QRS wave time point with which each respective coefficient is associated.

5. The device according to claim 1, wherein the composer is arranged to define a QRS fingerprint signal for each wavelet level of the wavelet transform, each based on a function of the coefficients derived by the extractor for a respective wavelet level.

6. The device according to claim 5, wherein the composer is arranged to apply a function selected from the group including the geometric median, the PCA or the ICA.

7. The device according to claim 5, wherein the composer is arranged to use a selected number of most recent coefficients in the buffer for each wavelet level.

8. The device according to claim 1, wherein the extractor is arranged to carry out a SWT-type wavelet transform.

9. A method for processing intracardiac signals comprising:
a) receiving electrocardiogram data and synchronised electrogram data,
b) analysing the electrocardiogram data to detect therein QRS wave time points,
c) carrying out a wavelet transform of the electrogram data,
d) deriving in the wavelet transform coefficients, each associated with a QRS wave time point detected in the operation b), and storing them in a buffer,
e) extracting from the buffer a QRS fingerprint signal and subtracting it in the wavelet transform of the operation c) at the QRS wave time points, and
f) carrying out an inverse wavelet transform of the signal of the operation e), and returning as output the corresponding denoised electrogram data.

10. The method according to claim 9, wherein the operation d) comprises deriving coefficients from the wavelet transform of the operation c) such that, for a selected coefficient corresponding to a given time point of a given level of the wavelet transform, the wavelet signal which corresponds to the given level of the wavelet transform and which is centred on the given time point has an overlap with a window extracted from the electrogram data from which the selected coefficient centred on the QRS wave time point with which the selected coefficient is associated, is derived.

11. The method according to claim 10, wherein the operation d) comprises weighting each derived coefficient before storing it in the buffer, according to the temporal overlap between the wavelet signal which corresponds to the level of the wavelet transform of each respective derived coefficient and which is centred on the time point corresponding to each respective derived coefficient and the window centred on the QRS wave time point associated with each respective derived coefficient.

12. The method according to claim 9, wherein the operation e) comprises defining a respective QRS fingerprint signal for each wavelet level of the wavelet transform of the operation b), each based on a function of the coefficients of the operation d) corresponding to a respective wavelet level.

13. The method according to claim 12, wherein the function is selected from the group including the geometric median, the PCA or the ICA.

14. A computer program product comprising a non-transitory computer readable medium storing a computer program comprising instructions which, when executed on processing circuitry, cause the processing circuitry to carry out the method according to claim 9.

15. (canceled)
